(19) United States
(12) Patent Application Publication
(10) Pub. No.: US 2025/0375395 A1
(43) Pub. Date: Dec. 11, 2025

(54) TRANSDERMAL FORMULATION AND DELIVERY METHOD OF LOW SOLUBILITY OR UNSTABLE UNIONIZED NEUTRAL DRUGS BY IN SITU SALT-TO-NEUTRAL DRUG CONVERSION OF SALT DRUG

(71) Applicant: Corium, LLC, Cambridge, MA (US)

(72) Inventors: Eun Soo Lee, Redwood City, CA (US); Amit K. Jain, Milpitas, CA (US); Parminder Singh, Union City, CA (US); Appala Sagi, Mountain View, CA (US)

(21) Appl. No.: 19/085,857

(22) Filed: Mar. 20, 2025

Related U.S. Application Data
(63) Continuation of application No. 15/660,935, filed on Jul. 26, 2017, now abandoned.
(60) Provisional application No. 62/367,542, filed on Jul. 27, 2016.
(60) Provisional application No. 62/367,502, filed on Jul. 27, 2016.
(60) Provisional application No. 62/423,133, filed on Nov. 16, 2016.
(60) Provisional application No. 62/444,763, filed on Jan. 10, 2017.
(60) Provisional application No. 62/444,745, filed on Jan. 10, 2017.
(60) Provisional application No. 62/457,794, filed on Feb. 10, 2017.
(60) Provisional application No. 62/504,408, filed on May 10, 2017.
(60) Provisional application No. 62/504,391, filed on May 10, 2017.

(51) Int. Cl. A61K 9/70; A61K 31/00; A61K 31/045; A61K 31/13; A61K 31/137; A61K 31/18; A61K 31/27; A61K 31/445; A61K 47/02; A61K 47/10; A61K 47/12; A61K 47/32; C08K 5/00

(52) U.S. Cl. CPC A61K 9/7038; A61K 9/7023; A61K 9/7053; A61K 9/7061; A61K 9/7084; A61K 9/7092; A61K 31/00; A61K 31/13; A61K 31/137; A61K 31/18; A61K 31/27; A61K 31/445; A61K 47/02; A61K 47/10; A61K 47/12; A61K 47/32; C08K 5/0016; A61K 31/045

(57) ABSTRACT

Compositions, devices, and methods for transdermal administration of active agents provided in their salt form instead of neutral form are provided.

### CROSS-REFERENCE TO RELATED APPLICATIONS

This application claims the benefit of U.S. Provisional Application No. 62/504,408, filed May 10, 2017; U.S. Provisional Application No. 62/504,391, filed May 10, 2017; U.S. Provisional Application No. 62/457,794, filed Feb. 10, 2017; U.S. Provisional Application No. 62/444,763, filed Jan. 10, 2017; U.S. Provisional Application No. 62/444,745, filed Jan. 10, 2017; U.S. Provisional Application No. 62/423, 133, filed Nov. 16, 2016; U.S. Provisional Application No. 62/367,542, filed Jul. 27, 2016 and U.S. Provisional Application No. 62/367,502, filed Jul. 27, 2016 each herein incorporated by reference in its entirety.

### TECHNICAL FIELD

The subject matter described herein relates to compositions, devices, and methods for transdermal administration of active agents provided in their salt form instead of neutral form.

### BACKGROUND

Active agents for transdermal delivery are typically provided in their neutral form because the neutral form is typically much more skin permeable than a corresponding salt form. In traditional transdermal formulations, a neutral form of an active agent is solubilized in a drug reservoir (also referred to sometimes as an adhesive matrix), and the active agent diffuses from the drug reservoir and into the skin. Transdermal patches, therefore, typically contain as much active agent dissolved in the drug reservoir as the agent's solubility in the components of the 23-30 allows, often with solubilizers to enhance its solubility. Alternatively, neutral, solid particles of active agent are sometimes dispersed in the drug reservoir, so long as the particles' dissolution rate is such that a constant supply of dissolved active agent is provided.

For many active agents, however, a neutral form is difficult to solubilize, formulate, and/or administer to a patient or subject. When a drug has a low solubility as a unionized neutral form in a drug reservoir, it is difficult to incorporate sufficient amount as a solubilized form to deliver at a therapeutic level for multiple days. Moreover, a dissolved active agent may recrystallize as large crystals during the process of solvation, coating, and drying. Further, many active agents are less stable in neutral form than in salt form.

There is a need in the art for improved compositions, devices, patches, systems, and methods for transdermal delivery of active agents that address these shortcomings.

### BRIEF SUMMARY

The following aspects and embodiments thereof described and illustrated below are meant to be exemplary and illustrative, not limiting in scope.

In one aspect, a composition for transdermal delivery is provided. The composition comprises a drug reservoir comprising a salt form of an active agent and a proton accepting and/or proton donating entity.

In one aspect, a composition for transdermal delivery is provided. The composition comprises a drug reservoir comprising an amine salt form of an active agent and a proton accepting entity.

In one embodiment, the active agent is donepezil, rivastigmine, memantine, fingolimod, or tamsulosin.

In one embodiment, the drug reservoir comprises between about 1-70% w/w of the active agent.

In another embodiment, the proton accepting entity is a proton accepting polymer.

In yet another embodiment, the proton accepting entity is an amine functionalized polystyrene miscrosphere or a dimethyl aminoethyl methacrylate-based acrylate.

In still another embodiment, the proton accepting entity is sodium bicarbonate. In other embodiments, the proton accepting entity excludes sodium bicarbonate.

In another embodiment, the drug reservoir comprises between about 0.5-35% w/w of the proton accepting entity.

In another aspect, a composition for transdermal delivery comprises a drug reservoir comprising an acid salt form of an active agent and a proton donating polymer.

In one embodiment, the active agent is an acid salt drug selected from the group consisting of sodium alendronate, tresprostinil sodium, sodium diclofenac, naproxen sodium, and ketoprofen sodium.

In one embodiment, the drug reservoir comprises between about 5-35% w/w of the active agent.

In another embodiment, the proton donating polymer is an anionic copolymer based on methacrylic acid or a carboxylated polystyrene microsphere.

In still another embodiment, the drug reservoir comprises between about 0.5-35% w/w of the proton donating polymer.

In yet another embodiment, the composition can further comprise a salt form solubilizer selected from the group consisting of water, alcohols, glycerol, propylene glycol, ethylene glycol, dimethyl sulfoxide, and N-methylpyrrolidone.

In one embodiment, the drug reservoir comprises up to 15% w/w of the salt form solubilizer.

In one embodiment, the composition can further comprise a neutral form solubilizer selected from the group consisting of a fatty acid ester, a dicarboxylic acid ester, a glycerol ester, a lactate, a fatty alcohol, sorbitan monolaurate, sorbitan monooleate, lauryl lactate, propylene glycol monolaurate, dimethyl succinate, lauryl alcohol, and oleyl alcohol.

In another embodiment, the drug reservoir comprises up to 15% w/w of the neutral form solubilizer.

In still another embodiment, the composition can comprises a plasticizer selected from the group consisting of a dicarboxylic acid ester, an adipate, a sebacate, a maleate, a tricarboxylic ester, triethyl citrate, tributyl citrate, a glycerol esters, and triacetin.

In yet another embodiment, the drug reservoir comprises up to 20% w/w of the plasticizer.

In another embodiment, the composition comprises an additive selected from the group consisting of crospovidone and colloidal silicone dioxide.

In another embodiment, the drug reservoir comprises up to 25% w/w of additive.

In another embodiment, the drug reservoir comprises an adhesive agent selected from the group consisting of an acrylate, polyisobutylene, silicone adhesive, and styrene block copolymer based adhesive.

In one embodiment, the drug reservoir comprises up to 65% w/w of the adhesive agent.

In another aspect, a transdermal patch comprising a drug reservoir layer as described herein, a backing layer, and a contact adhesive layer is provided.

In one embodiment, the backing layer is an occlusive polymer film.

In one embodiment, the contact adhesive layer comprises an adhesive selected from the group consisting of an acrylate, polyisobutylene, silicone adhesive, and styrene block copolymer based adhesive.

In one embodiment, the transdermal patch further comprises a nonwoven tie layer between the drug reservoir and the contact adhesive layer.

In one embodiment, the transdermal patch further comprises a rate-controlling membrane between the drug reservoir and the contact adhesive layer.

In one embodiment, the transdermal patch comprises at least two drug reservoir layers.

In another embodiment, the at least two drug reservoir layers are separated by a nonwoven tie layer.

### BRIEF DESCRIPTION OF THE FIGURES

FIGS. 1-3 are illustrations of exemplary embodiments of transdermal patch configurations.

FIG. 4 is a graph of average skin flux for donepezil transdermal delivery devices, in μg/cm²·hr, in vitro as a function of time, in hours, in an in vitro skin permeation test for devices having formulations according to Example 1 (squares), Example 2 (triangles), and Example 7 (circles).

FIG. 5 is a graph of average skin flux for memantine transdermal delivery devices, in μg/cm²·hr, in vitro as a function of time, in hours, in an in vitro skin permeation test for devices having a formulation according to Example 4.

### DETAILED DESCRIPTION

### I. DEFINITIONS

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Compositions, devices, and methods described herein are not limited to the specific polymers, excipients, cross-linking agents, additives, manufacturing processes, or adhesive products described herein. It will be understood that the particular terminology used herein is for the purpose of describing particular embodiments and is not intended to be limiting.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to a “solvent” includes a single solvent as well as two or more of the same or different solvents, and the like.

The use of terms of order or importance, including “first” and “second,” is to distinguish and identify individual elements and does not denote or imply a particular order or importance unless clearly indicated by context.

The term “active agent” as used herein refers to a chemical material or compound suitable for topical or transdermal administration and that induces a desired effect. The terms include agents that are therapeutically effective, prophylactically effective, and cosmetically effective agents. The terms “active agent,” “drug,” and “therapeutic agent” are used interchangeably herein.

An “adhesive matrix” as described herein includes matrices made in one piece, for example, matrices made via solvent casting or extrusion as well as matrices formed in two or more portions that are then pressed or joined together.

The term “skin” as used herein refers to skin or mucosal tissue, including the interior surface of body cavities that have a mucosal lining. The term “skin” should be interpreted as including “mucosal tissue” and vice versa.

The term “therapeutically effective amount” as used herein refers to the amount of an active agent that is nontoxic but sufficient to provide the desired therapeutic effect. The amount that is “effective” will vary from subject to subject, depending on the age and general condition of the individual, the particular active agent or agents, and the like as known to those skilled in the art.

The terms “transdermal” or “transdermal delivery” as used herein refer to administration of an active agent to a body surface of an individual so that the agent passes through the body surface (e.g., through the skin) and into the individual's blood stream. The term “transdermal” is intended to include transmucosal administration, i.e., administration of a drug to the mucosal (e.g., sublingual, buccal, vaginal, rectal, etc.) surface of an individual so that the agent passes through the mucosal tissue and into the individual's blood stream.

### II. COMPOSITIONS/DEVICES

Compositions and/or devices are provided for transdermal administration of active agents. Compositions may be used in devices, patches, and/or systems for transdermal delivery of one or more active agents. Compositions described herein are contemplated for use in transdermal delivery systems, devices, patches, and/or methods as described herein.

Active agents for transdermal delivery are typically provided in their neutral form because the neutral form is typically much more skin permeable than a corresponding salt form. Many active agents, however, are difficult to solubilize in a sufficient amount in a neutral form, difficult to administer in a neutral form at a steady rate for multiple days, and/or less stable in a neutral form than a salt form. As such, the present invention encompasses the recognition that certain active agents are better suited for administration in their salt forms.

In general, compositions described herein provide an active agent in its salt form and at least one proton donating or proton accepting entity. The proton donating or proton accepting entity promotes conversion of the active agent from a salt to its neutral form to improve skin permeability of the active agent. In some embodiments, compositions contain one or more additional ingredients (for example, solubilizers, plasticizers, matrix modifying additives, and/or adhesives). In some embodiments, active agent compositions described herein can be provided in the form of a transdermal drug delivery device, such as a patch.

### A. Compositions for Transdermal Delivery of Active Agents

In a first aspect, compositions comprising a layer or matrix comprised of an adhesive, a salt form of at least one active agent and at least one proton accepting or proton donating entity are provided. In general, the salt form of a provided active agent will react with a provided proton accepting or proton donating entity in order to generate a neutral form of the active agent that is more skin permeable than the salt form. In some embodiments, the adhesive composition may include one or more additional ingredients that cause the neutral form of the active agent to be generated at a specified and/or desired rate. Such compositions can provide, in some embodiments, a relatively constant activity of an active agent.

The compositions may further include one or more of the following: one salt form solubilizer (total 0-50% w/w), one neutral form solubilizer (total 0-50% w/w), one plasticizer for a proton accepting entity and/or proton donating entity (total 0-50% w/w), matrix modifying additives (total 0-50% w/w), and adhesive polymers (total 0-95% w/w).

It will be appreciated that all w/w % or wt % described herein may refer to wet or dry weight of the composition.

In some embodiments, compositions comprise micronized particles of a salt form of one or more active agents dispersed in an adhesive matrix or a drug reservoir. In some embodiments, the salt form of at least one active agent is present in the drug reservoir in an amount between about 1-70 wt %, about 1-50 wt %, about 1-35 wt %, about 1-25 wt %, about 2-70 wt %, about 2-50 wt %, about 2-35 wt %, about 5-70 wt %, about 5-50 wt %, about 5-35 wt %, about 5-30 wt %, about 5-25 wt %, about 5-20 wt %, about 5-15 wt %, about 5-10 wt %, about 10-35 wt %, about 10-30 wt %, about 10-25 wt %, about 10-20 wt %, about 10-15 wt %, about 20-35 wt %, about 20-30 wt %, about 20-25 wt %, about 25-35 wt %, about 25-30 wt %, or about 30-35 wt %.

In some embodiments, the drug reservoir further comprises a solubilizer that has a limited solubility for the salt form of an active agent (the “salt form solubilizer”). In some embodiments, the micronized particles of a salt form of an active agent will ionize in the salt form solubilizer. In some embodiments, the micronized salt form particles will be maintained in equilibrium with the dissolved, ionized salt form. In some embodiments, the salt form solubilizer has only a limited degree of solubility for the micronized salt particles, such that the equilibrium favors the micronized salt particles over the dissolved, ionized salt form.

In some embodiments, a salt form solubilizer has a solubility for the salt of at least about 0.1% w/w, at least about 0.2% w/w, at least about 0.3% w/w, at least about 0.4% w/w, at least about 0.5% w/w, or at least about 1.0% w/w. In some embodiments, the salt form solubilizer has a solubility for the salt of less than about 30% w/w or less than about 25% w/w or 20% w/w.

In some embodiments, a salt form solubilizer is a protic solvent (e.g., a solvent that has a hydrogen atom bound to an oxygen (e.g., as in a hydroxyl group) or a nitrogen (e.g., as in an amine group), and/or any solvent that contains labile protons). Exemplary salt form solubilizers include, but are not limited to, water, alcohols (e.g., ethanol, methanol, etc.), glycerol, propylene glycol, ethylene glycol, dimethyl sulfoxide, N-methylpyrrolidone, and/or combinations thereof.

In some embodiments, the drug reservoir comprises about 0-50 wt %, about 0-20 wt %, about 0-10 wt %, about 0-5 wt %, about 1-50 wt %, about 1-20 wt %, about 2-50 wt %, about 2-20 wt %, about 5-50 wt %, about 5-20 wt %, about 5-15 wt %, about 5-10 wt %, or about 10-15 wt % of at least one salt form solubilizer.

In some embodiments, the dissolved, ionized salt form will react with a proton accepting entity (for amine drug salt) or a proton donating entity (for carboxylic drug salt), also provided in the matrix. In general, proton accepting entities have primary, secondary, or tertiary amine groups. In general, proton donating entities have carboxylic groups. In some embodiments, proton accepting entities and/or proton donating entities are dissolved and intermixed with the drug reservoir. In some embodiments, proton accepting entities and/or proton donating entities are dispersed as fine particles. In some embodiments, proton accepting entities and/or proton donating entities are plasticized.

In some embodiments, a drug reservoir comprises about 0.5-30 wt %, about 1-30 wt %, about 5-30 wt %, about 10-30 wt %, about 15-30 wt %, about 20-30 wt %, about 25-30 wt %, about 0.5-25 wt %, about 1-25 wt %, about 5-25 wt %, about 10-25 wt %, about 15-25 wt %, about 20-25 wt %, about 0.5-20 wt %, about 1-20 wt %, about 5-20 wt %, about 10-20 wt %, about 15-20 wt %, about 0.5-15 wt %, about 1-15 wt %, about 5-15 wt %, about 10-15 wt %, about 0.5-10 wt %, about 1-10 wt %, about 5-10 wt %, about 0.5-5 wt %, or about 1-5 wt % of at least one proton accepting entity or at least one proton donating entity.

In some embodiments, a proton accepting entity or proton donating entity is a polymer. In some embodiments, proton accepting entities are amine-functional polymers. Exemplary proton accepting polymers include, but are not limited to, amine-functionalized polystyrene miscrosphere, dimethyl aminoethyl methacrylate based acrylates (e.g., EUDRAGIT® EPO or EUDRAGIT® E100), and/or combinations thereof. In some embodiments, proton donating entities are carboxylic-functional polymers. Exemplary proton donating polymers include, but are not limited to, anionic copolymers based on methacrylic acid and polyacrylic acid (e.g., EUDRAGIT® L100, EUDRAGIT® L100-55, EUDRAGIT® S100), carboxylated polystyrene microsphere, and/or combinations thereof.

In some embodiments, the reaction of a salt form of an active agent with a proton accepting polymer or proton donating polymer will generate a skin permeable, neutral form of the active agent and a solid polymer. As the neutral active agent is depleted (by diffusion into the skin) and the polymer becomes separated as a solid phase in the matrix, the reaction continues to move forward, maintaining a relatively steady concentration and/or activity of the neutral form.

In some embodiments, the proton accepting entity for an amine drug salt is an inorganic salt with mild basicity whose pKa is lower than that of the amine drug salt. For example, sodium bicarbonate has pKa=6.4 which is lower than most amine salt drugs. In some embodiments, the proton accepting entity for an amine drug salt is a sodium bicarbonate. In such embodiments, the reaction products include a skin permeable, neutral form of the active agent, water, and carbon dioxide. As the neutral active agent and CO₂ are depleted (by diffusion into the skin and escaped gas, respectively), the reaction continues to move forward, maintaining a relatively steady concentration and/or activity of the neutral form.

In some embodiments, compositions further comprise one or more solubilizers for the neutral form of the active agent (a “neutral form solubilizer”). In some embodiments, the neutral form solubilizer helps ensure that a neutral active agent, once formed, can persist long enough to diffuse into the skin.

In some embodiments, a neutral form solubilizer has a solubility for the neutral form of the active agent of at least about 0.1% w/w. In some embodiments, the neutral form solubilizer has a solubility for the neutral form of the active agent of less than about 30% w/w.

In some embodiments, exemplary neutral form solubilizers generally include, but are not limited to, fatty acid esters, lactate esters, dicarboxylic esters, citrate esters, glycerol esters, fatty alcohols, and/or combinations thereof. In some embodiments, exemplary neutral form solubilizers include, but are not limited to, sorbitan monooleate, sorbitan monolaurate (Span® 20), propylene glycol monolaurate, lauryl lactate, dimethyl succinate, triethyl citrate, triacetin, lauryl alcohols, oleyl alcohols, and/or combinations thereof.

In some embodiments, the drug reservoir comprises about 0-40 wt %, about 0-30 wt %, about 0-20 wt %, about 0-15 wt %, about 0-10 wt %, about 0-5 wt %, about 1-40 wt %, about 1-30 wt %, about 1-20 wt %, about 2-40 wt %, about 2-30 wt %, about 2-20 wt %, about 5-20 wt %, about 1-15 wt %, about 2-15 wt %, about 5-15 wt %, about 5-10 wt %, or about 10-15 wt % of at least one neutral form solubilizer.

In some embodiments, active agents include amine salt drugs or acid salt drugs.

In some embodiments, the active agent is an amine salt drug. In some embodiments, amine salt drugs have a solubility of at least about 0.1 mg/g, at least about 0.2 mg/g, at least about 0.3 mg/g, at least about 0.4 mg/g, at least about 0.5 mg/g, or at least about 1.0 mg/g in the drug reservoir. In some embodiments, amine salt drugs have a solubility of less than about 100 mg/g in the drug reservoir. Exemplary amine salt drugs include, but are not limited to, donepezil, rivastigmine, memantine, tamsulosin, rotigotine, fentanyl, escitalopram, ropinirole, pramipexole, buprenorphine, fingolimod and lidocaine.

In some embodiments, a micronized amine salt form of an active agent is dispersed in a matrix that further comprises at least one salt form solubilizer. The salt form of the active agent (“DHCl”) is partially dissolved and ionized, as shown in Equation 1:

$$DH Cl {\to }^{Ionized} {DH}^{+} + {Cl}^{-} \qquad ( 1 )$$

In some embodiments, the dissolved, ionized active agent (“DH”) interacts with a proton acceptor amine polymer (“PolymerN”) as shown in Equation 2. As the neutral form of the active agent (“D”) is depleted by diffusion, the reaction moves toward continuous formation of D.

$${{DH}^{+} +}_{{Dissolved \& skin}_{{impermeable}_{amine salt form}}} {{Polymer N}_{}}_{Solid polymer} + {Cl}^{-} {\to }^{Equilibrium Constant = K} {Polymer NHCl}_{{}_{Solid polymer}} {+ D}_{{Dissolved \& skin}_{permeable neutral}} \qquad ( 2 )$$

In some embodiments, the reaction byproduct (“Polymer NHCl”) is sequestered as solid phase in the matrix. In some embodiments, the neutral form of the active agent (“D”) is depleted by diffusion through into the skin.

Because a proton is sequestered from DH⁺, the dissolved, ionized active agent in the equilibrium reaction (2), the equilibrium constant is expressed as in Equation 3:

$$K = \frac{[ D ]}{[ {DH}^{+} ]} \qquad ( 3 )$$

In some embodiments, a basic inorganic salt whose pKa is lower than an amine salt drug can also be used to generate a neutral form of the drug. For example carbonic acid/sodium bicarbonate has pKa=6.4. In some embodiments, sodium bicarbonate can be used as a proton accepting entity for an amine salt drug. In one embodiment, sodium bisulfite is the protein accepting entity for an amine salt drug.

Ionization of sodium bicarbonate is shown in Equation 5, which generates a sodium ion and a bicarbonate ion:

$${NaHCO}_{3} {\to }^{Ionization} {Na}^{+} + {HCO}_{3}^{-} \qquad ( 5 )$$

The salt form of an active agent (“DHCl”) is partially dissolved and ionized, as shown in Equation 6:

$${DH}^{+} + {Cl}^{-} \qquad ( 6 )$$

In some embodiments, a bicarbonate ion accepts a proton from the amine salt drug to form carbonic acid. Carbonic acid decomposes into water and carbon dioxide, which escapes from the patch. As carbon dioxide escapes and the neutral active agent “D” is depleted by diffusion into the skin, the equilibrium of Equation (8) moves toward the right (continuous formation of neutral drug “D”).

$${DH}^{+} + {HCO}_{3}^{-} + NaCl {\to }^{Equilibrium Constant = K} D + {H}_{2} {CO}_{3} + NaCl - {\to }^{Further reaction} D + {H}_{2} O + NaCl ↓ + {CO}_{2} ↑ ( {CO}_{2} escapes as gas ) \qquad ( 8 )$$

$$K = \frac{[ D ]}{[ {DH}^{+} ] [ {HCO}_{3}^{-} ]} \qquad ( 8 )$$

In some embodiments, the active agent is an acid salt drug. In some embodiments, acid salt drugs have a solubility of at least about 0.1 mg/g, at least about 0.2 mg/g, at least about 0.3 mg/g, at least about 0.4 mg/g, at least about 0.5 mg/g, or at least about 1.0 mg/g in the drug reservoir. In some embodiments, acid salt drugs have a solubility of less than about 100 mg/g in the drug reservoir. Exemplary acid salt drugs include, but are not limited to sodium alendronate, tresprostinyl sodium, sodium diclofenac, ketoprofen sodium, etc.

In some embodiments, a micronized acid salt form of an active agent is dispersed in a matrix that further comprises at least one salt form solubilizer. The salt form of the active agent (“ANa”) is partially dissolved and ionized, as shown in Equation 9:

$$A Na {\to }^{Ionized} {A}^{-} + {Na}^{+} \qquad ( 9 )$$

In some embodiments, the dissolved, ionized active agent (“A”) interacts with a proton donor polymer (“PolymerCOOH”) as shown in Equation 10. As the neutral form of the active agent (“AH”) is depleted by diffusion, the reaction moves toward continuous formation of AH.

$${{A}^{-} +}_{{Dissolved \& skin}_{{impermeable acid}_{salt form}}} {{Polymer COOH}_{}}_{Solid polymer} + {Na}^{-} {\to }^{Equilibrium Constant = K} {Polymer COONa}_{{}_{Solid polymer}} {+ AH}_{{Dissolved \& skin}_{{permeable neutral}_{form}}} \qquad ( 10 )$$

In some embodiments, the reaction byproduct (“Polymer COONa”) is sequestered as solid phase in the matrix. In some embodiments, the neutral form of the active agent (“AH”) is depleted by diffusion through into the skin.

Because the reaction byproduct is sequestered as a solid phase in Equation 10, the equilibrium constant is expressed as in Equation 11:

$$K = \frac{[ AH ]}{[ {A}^{-} ]} \qquad ( 11 )$$

In some embodiments, a composition for transdermal delivery of an active agent further comprises one or more plasticizers for the proton accepting or proton donating entity. In some embodiments, salt form solubilizers and/or neutral form solubilizers already present in the composition may also serve as plasticizers for the proton accepting or proton donating entities. In some embodiments, a plasticizer that does not also serve as a salt form and/or neutral form solubilizer is included in a composition. Exemplary plasticizers include, but are not limited to, dicarboxylic acid esters (e.g., adipates, sebacates, maleates, etc.), tricarboxylic esters (e.g., triethyl citrate, tributyl citrate, etc.), esters of glycerol (e.g., triacetin, etc.), and/or combinations thereof.

In some embodiments, an adhesive matrix or drug reservoir comprises about 0-20 wt %, about 0-15 wt %, about 0-10 wt %, about 0-5 wt %, about 5-20 wt %, about 5-15 wt %, about 5-10 wt %, about 10-20 wt %, about 10-15 wt %, or about 15-20 wt % of at least one plasticizer.

In some embodiments, a composition for transdermal delivery of an active agent further comprises at least one additive, also referred to as a matrix modifying additive. In some embodiments, matrix modifying additives modify cohesion and/or diffusivity of described active agent compositions. In some embodiments, matrix modifying additives can absorb moisture and/or water emanating from the skin under occlusion, which improves adhesion to the skin. In some embodiments, matrix modifying additives facilitate homogenization of the drug reservoir. Exemplary matrix modifying additives include, but are not limited to, crospovidone (KOLLIDON® CL-M, etc.), cross-linked polyvinylpyrrolidone (PVP), a soluble polyvinylpyrrolidone (PVP), fumed silica, colloidal silicone dioxide, a cellulose derivative (e.g. hydroxypropyl cellulose (HPC), hydroxyethylcellulose (HEC)), a polyacrylamide, a polyacrylic acid, a polyacrylic acid salt, a clay (e.g., kaolin, bentonite, etc.), fumed silica, and/or combinations thereof. An exemplary commercial fumed silica product is AEROSIL® 200P, an amorphous, anhydrous colloidal silicon dioxide (Evonik Industries). Another exemplary fumed silica product is Cab-O-Sil® (Cabot Corporation, Boston, Mass.).

In some embodiments, the drug reservoir comprises about 0-25 wt %, about 0-20 wt %, about 0-15 wt %, about 0-10 wt %, about 0-5 wt %, about 5-25 wt %, about 5-20 wt %, about 5-15 wt %, about 5-10 wt %, about 10-25 wt %, about 10-20 wt %, about 10-15 wt %, about 15-25 wt %, about 15-20 wt %, or about 20-25 wt % of at least one matrix modifying additive.

In some embodiments, a composition for transdermal delivery of an active agent further comprises an adhesive or adhesive polymer. Exemplary adhesives and adhesive polymers include, but are not limited to, acrylates, polyisobutylene, silicone adhesive, styrene block copolymer based adhesives, and/or combinations thereof. In some embodiments, proton accepting and/or proton donating polymers are film-formable in the presence of salt form and/or neutral form solubilizers. In such embodiments, compositions may not require a separate adhesive polymer.

In some embodiments, the drug reservoir comprises about 0-65 wt %, about 0-60 wt %, about 0-55 wt %, about 0-50 wt %, about 0-45 wt %, about 0-40 wt %, about 0-35 wt %, about 0-30 wt %, about 0-25 wt %, about 0-20 wt %, about 0-15 wt %, about 0-10 wt %, about 0-5 wt %, 5-65 wt %, about 5-60 wt %, about 5-55 wt %, about 5-50 wt %, about 5-45 wt %, about 5-40 wt %, about 5-35 wt %, about 5-30 wt %, about 5-25 wt %, about 5-20 wt %, about 5-15 wt %, about 5-10 wt %, 10-65 wt %, about 10-60 wt %, about 10-55 wt %, about 10-50 wt %, about 10-45 wt %, about 10-40 wt %, about 10-35 wt %, about 10-30 wt %, about 10-25 wt %, about 10-20 wt %, about 10-15 wt %, 15-65 wt %, about 15-60 wt %, about 15-55 wt %, about 15-50 wt %, about 15-45 wt %, about 15-40 wt %, about 15-35 wt %, about 15-30 wt %, about 15-25 wt %, about 15-20 wt %, 20-65 wt %, about 20-60 wt %, about 20-55 wt %, about 20-50 wt %, about 20-45 wt %, about 20-40 wt %, about 20-35 wt %, about 20-30 wt %, about 20-25 wt %, 25-65 wt %, about 25-60 wt %, about 25-55 wt %, about 25-50 wt %, about 25-45 wt %, about 25-40 wt %, about 25-35 wt %, about 25-30 wt %, 30-65 wt %, about 30-60 wt %, about 30-55 wt %, about 30-50 wt %, about 30-45 wt %, about 30-40 wt %, about 30-35 wt %, 35-65 wt %, about 35-60 wt %, about 35-55 wt %, about 35-50 wt %, about 35-45 wt %, about 35-40 wt %, 40-65 wt %, about 40-60 wt %, about 40-55 wt %, about 40-50 wt %, about 40-45 wt %, 45-65 wt %, about 45-60 wt %, about 45-55 wt %, about 45-50 wt %, 50-65 wt %, about 50-60 wt %, about 50-55 wt %, 55-65 wt %, about 55-60 wt %, or about 60-65 wt % of at least one adhesive polymer.

The composition may also include other conventional additives such as adhesive agents, antioxidants, crosslinking agents, curing agents, pH regulators, pigments, dyes, refractive particles, conductive species, antimicrobial agents, opacifiers, gelling agents, viscosity modifiers, thickening agents, stabilizing agents, and the like as known in the art. In those embodiments wherein adhesion needs to be reduced or eliminated, conventional detackifying agents may also be used. In some embodiments, agents such as antimicrobial agents are included to prevent spoilage upon storage, e.g., to inhibit growth of microbes such as yeasts and molds. Suitable antimicrobial agents are typically selected from the group consisting of the methyl and propyl esters of p-hydroxybenzoic acid (e.g., methyl and propyl paraben), sodium benzoate, sorbic acid, imidurea, and/or combinations thereof. These additives, and amounts thereof, are selected in such a way that they do not significantly interfere with the desired chemical and physical properties of the adhesive and/or active agent.

Compositions may also contain irritation-mitigating additives to minimize or eliminate the possibility of skin irritation and/or skin damage resulting from the active agent, the proton donating or proton accepting entity, salt form solubilizer, neutral form solubilizer, plasticizer, matrix modifying additive, adhesive and/or other components of the composition. Suitable irritation-mitigating additives include, for example: corticosteroids; a-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine; and/or combinations thereof.

Methods for preparing or manufacturing active agent compositions are also provided. Exemplary methods are set forth in the Examples section. Methods for preparing active agent compositions generally involve mixing an active agent at 5-35% w/w with a proton accepting or proton donating entity at 0.5-30% w/w, optionally along with a salt form solubilizer at 0-15% w/w, a neutral form solubilizer at 0-15% w/w, a matrix modifying additive at 0-25% w/w, a plasticizer at 0-20% w/w, an adhesive polymer at 0-65% w/w, and/or combinations thereof.

### B. Transdermal Devices

In certain aspects, the compositions described herein are provided in transdermal devices (e.g., patches). In general, transdermal patches comprise a backing layer, at least one drug reservoir, and a contact adhesive layer. It will be appreciated that the drug reservoir and contact adhesive layer can be in the form of a single layer or matrix comprised of an adhesive and a drug. In some embodiments, transdermal patches further comprise one or more release liners, tie layers, rate-controlling membranes, and/or various combinations of the foregoing.

In some embodiments, transdermal patches comprise one or more of the following components: backing layer, drug reservoir, contact adhesive layer, release liner, tie layer, rate-controlling membrane, and/or various combinations of the foregoing.

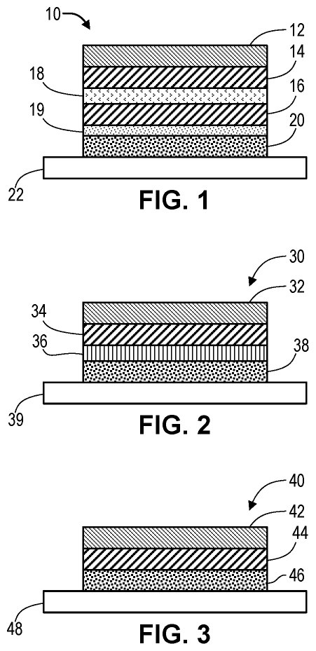

Exemplary transdermal patches are shown in FIGS. 1-3. FIG. 1 shows an exemplary transdermal patch 10 comprising a backing layer 12, multiple drug reservoirs 14, 16 separated by a nonwoven tie layer or a rate controlling membrane 18, a contact adhesive layer 20, and a release liner 22. This particular example presents multiple drug reservoirs separated by a tie layer or a rate controlling membrane 18, but in some embodiments, multiple adhesive layers may be in direct contact with each other without a tie layer, such as optional tie layer 19. In such embodiments wherein the transdermal patch comprises multiple drug reservoirs, each drug reservoir may comprise the same or different active agents. In such embodiments wherein the transdermal patch comprises multiple drug reservoirs, each drug reservoir may comprise different concentrations of the same active agent.

FIG. 2 shows an exemplary transdermal patch 30 comprising a backing layer 32, a drug reservoir 34, a tie layer or rate-controlling membrane 36, a contact adhesive layer 38, and a release liner 39.

FIG. 3 shows an exemplary transdermal patch 40 comprising a backing layer 42, a drug reservoir 44, a contact adhesive layer 46, and a release liner 48.

In some embodiments, a backing layer provides a structural element for holding or supporting the adhesive layer. A backing layer may be formed of any suitable material as known in the art. In some embodiments, a backing layer is occlusive. In some embodiments, a backing layer is preferably impermeable or substantially impermeable to moisture. In one exemplary embodiment, the barrier layer has an MVTR (moisture vapor transmission rate) of less than about 50 g/m²-day. In some embodiments, a backing layer is preferably inert and/or does not absorb components of the adhesive layer, including the active agent. In some embodiments, a backing layer preferably prevents release of components of the adhesive layer through the backing layer. A backing layer may be flexible or nonflexible. A backing layer is preferably at least partially flexible such that the backing layer is able to conform at least partially to the shape of the skin where the patch is applied. In some embodiments, a backing layer is flexible such that the backing layer conforms to the shape of the skin where the patch is applied. In some embodiments, a backing layer is sufficiently flexible to maintain contact at the application site with movement, e.g., skin movement. Typically, the material used for a backing layer should permit the device to follow the contours of the skin or other application site and be worn comfortably on areas of skin such as at joints or other points of flexure, that are normally subjected to mechanical strain with little or no likelihood of the device disengaging from the skin due to differences in the flexibility or resiliency of the skin and the device.

In some embodiments, a backing layer is formed of one or more of a film, non-woven fabric, woven fabric, laminate, and combinations thereof. In some embodiments, the film is a polymer film comprised of one or more polymers. Suitable polymers are known in the art and include, but are not limited to, elastomers, polyesters, polyethylene, polypropylene, polyurethanes, polyether amides, and/or combinations thereof. In some embodiments, a backing layer is formed of one or more of polyethylene terephthalate, various nylons, polypropylene, metalized polyester films, polyvinylidene chloride, aluminum foil, and/or combinations thereof. In some embodiments, a backing layer is a fabric formed of one or more of polyesters such as polyethylene terephthalate, polyurethane, polyvinyl acetate, polyvinylidene chloride, polyethylene, and/or combinations thereof. In one particular, but non-limiting embodiment, the backing layer is formed of a polyester film laminate. Exemplary particular polyester film laminates include, but are not limited to, the polyethylene and/or polyester laminates such as those sold under the names Scotchpak™ #9723, Scotchpak™ #1012, and the like.

In some embodiments, the drug reservoir generally comprises a salt form of an active ingredient(s) (total 5-35% w/w), at least one salt form solubilizer (total 0-20% w/w), at least one neutral form solubilizer (total 0-20% w/w), at least one proton accepting entity and/or proton donating entity (total 0.5-30% w/w), optionally at least one plasticizer for a proton accepting entity and/or proton donating entity (total 0-20% w/w), matrix modifying additives (total 0-25% w/w), and optionally adhesive polymers (total 0-65% w/w). In some embodiments, the drug reservoir comprises any of the compositions for transdermal delivery described herein, e.g., in the Examples and in Section II.A.

In general, a tie layer comprises a nonwoven fabric and/or a rate controlling polymer membrane.

In some embodiments, devices further include one or more fabric or tie layers within or between the drug reservoir layers. It will be appreciated that a tie layer may be included between one, some, or all of the layers. In some embodiments, a tie layer is useful to increase bonding between layers of the device. Tie layers may increase bonding by providing chemical groups for the polymers to bind. In some embodiments, a tie layer is useful as a separation for adhesive matrix layers.

In some embodiments, a tie layer does not affect the rate of release of an active agent from the adhesive layers. In some embodiments, tie layers may comprise nonwoven films that include, but are not limited to, nonwoven polyesters (e.g., REEMAY®), porous polyethylene film (DELNET®), nylon, cotton, and the like, and/or combinations thereof.

In some embodiments, tie layers may comprise rate controlling polymer membranes. Exemplary rate controlling polymer membranes include, but are not limited to, microporous polymer films such as CELGARD® 2400 (microporous polypropylene), polyethylenes (e.g., microporous polyethylene), vinyl acetate polymers and copolymers, and the like, and/or combinations thereof. In general, a rate controlling polymer membrane allows for a rate-controlled release of the drug from the drug reservoir layer.

In some embodiments, the tie layer comprises a nonwoven fabric and does not comprise a rate controlling polymer membrane. In some embodiments, a tie layer comprises a rate controlling polymer membrane and does not comprise a nonwoven fabric. In some embodiments, a tie layer comprises both a nonwoven fabric and a rate controlling polymer membrane. To give but one example, a nonwoven fabric and a rate controlling polymer membrane may both be used when the tie layer is embedded within the drug reservoir to help improve drug reservoir cohesion (see, e.g., FIG. 1).

The device includes at least one adhesive layer. In embodiments, at least one of the adhesive layers is an adhesive matrix comprising one or more active agents as described below. The adhesive layer adheres to a drug reservoir, an adjacent adhesive layer, a tie layer, a release liner, and/or skin at the administration site. In some embodiments, an adhesive layer serves to release the active agent to the skin. In some embodiments, one or more of the drug reservoir adhesive and/or the contact layer adhesive are formed of an adhesive matrix. Exemplary adhesives include, but are not limited to, acrylates, polyisobutylene, a silicone adhesive, a styrene block copolymer based adhesive, or the like, and/or combinations thereof.

In some embodiments, the drug reservoir of the delivery system provides an in vitro skin flux of an active agent between about 0.5-30 μg/cm²-hr for a period of at least about 2 days.

In embodiments, a release liner is at least partially in contact with at least one of the adhesive layers to protect the adhesive layer(s) prior to application. A release liner is typically a disposable layer that is removed prior to application of the device to the treatment site. In some embodiments, a release liner does not absorb components of the adhesive layer(s), including the active agent. In some embodiments, a release liner is impermeable to components of the adhesive layer(s) (including the active agent) and prevents release of components of the adhesive layer(s) through the release liner. In some embodiments, a release liner is formed of one or more of a film, non-woven fabric, woven fabric, laminate, and/or combinations thereof. In some embodiments, a release liner is a silicone-coated polymer film or paper. In some non-limiting embodiments, a release liner is a silicone-coated polyethylene terephthalate (PET) film, a fluorocarbon film, a fluorocarbon coated PET film, and/or combinations thereof.

Transdermal devices and systems (e.g., patches) may be prepared by any suitable methods as known in the art. In some general embodiments, transdermal devices are prepared by coating an appropriate amount of an adhesive polymer composition (with or without an active agent) onto a substrate such as a release liner or a backing layer. In some embodiments, the adhesive polymer composition is coated onto the release liner. In some embodiments, the adhesive polymer composition is coated onto the substrate or liner to a desired thickness. The thickness and/or size of the device and/or drug reservoir may be determined by one skilled in the art based at least on considerations of wearability and/or required dose. It will be appreciated that the administration site for the device will affect the wearability considerations due to the available size of the administration site and the use of the administration site (e.g., need for flexibility to support movement). In some embodiments, the device and/or drug reservoir has a thickness of between about 25-500 μm. The adhesive polymer composition and substrate are at least partially dried to remove any solvents. A release liner or backing layer is applied to the opposite side of the substrate. Where the substrate is not a release liner or backing layer, the substrate is replaced with the appropriate release liner or substrate. In embodiments that include multiple adhesive polymer layers, a first adhesive polymer composition is applied or coated onto the substrate, a tie layer material is applied to the formulation, and the second adhesive polymer composition is applied to the tie layer material. Adhesive polymer compositions and tie layers are laminated using any suitable methods known in the art. In some embodiments, adhesive layers are coated onto separate substrates or liners and then joined to form the transdermal delivery device. Where the delivery device includes a reservoir adhesive layer and a contact adhesive layer, adhesive polymer compositions may be coated onto the substrate or liner and laminated. It will be appreciated that any or all of the adhesive polymer composition layers may be dried before laminating the layers.

### III. METHODS OF TREATMENT

In another aspect, methods of treating a disease, condition, and/or disorder by transdermal administration of at least one active agent by the transdermal compositions, devices, and/or systems described herein are provided, and several non-limiting exemplary embodiments are set forth.

In some embodiments, compositions comprise methylphenidate (RITALIN®) as an active agent and are used for treating attention deficit hyperactivity disorder (ADHD), narcolepsy, and/or depression, e.g., through administration of methylphenidate by a transdermal patch. The FDA has approved doses of methylphenidate of 2.5 mg, 5 mg, 10 mg, 15 mg, 18 mg, 20 mg, 27 mg, 30 mg, 36 mg, 40 mg, 50 mg, 54 mg, and 60 mg.

In other embodiments, compositions as described herein comprising donepezil (ARICEPT®) as an active agent are used for treating Alzheimer's disease, e.g., through administration of donepezil by a transdermal patch. The FDA has approved doses of donepezil of 5 mg, 7 mg, 10 mg, 14 mg, and 23 mg. In some embodiments, compositions described herein allow for administration of doses that correspond to FDA approved doses.

In some embodiments, compositions as described herein comprising rivastigmine (Exelon®) as an active agent are used for treating Alzheimer's disease and/or Parkinson's disease dementia, e.g., through administration of rivastigmine by a transdermal patch. The FDA has approved daily doses of rivastigmine of 1.5 mg, 2.0 mg, 3.0 mg, 4.5 mg, 4.6 mg, 6.0 mg, 9.0 mg, 9.5 mg, and 13.3 mg. In some embodiments, compositions as described herein comprising memantine as an active agent are used for treating Alzheimer's disease, obsessive compulsive disorder, anxiety disorder, ADHD, and opioid dependence, e.g., through administration of memantine by a transdermal patch. The FDA has approved doses of memantine of 2 mg, 5 mg, 7 mg, 10 mg, 14 mg, 21 mg, and 28 mg.

In some embodiments, compositions as described herein comprising tamsulosin as an active agent are used for treating benign prostatic hyperplasia and/or acute urinary retention, e.g., through administration of tamsulosin by a transdermal patch. The FDA has approved doses of tamsulosin of 0.4 mg and 0.5 mg.

Transdermal compositions, devices, and/or systems described herein may be designed for long term use and/or continuous administration of at least one active agent. It will be appreciated that the total dose of the active agent per transdermal device will be determined by the nature of the active agent(s), the size of the device, and/or the loading of the active agent within the drug reservoir. In some embodiments, the application period for the transdermal device is between about 1-10 days, 1-7 days, 1-5 days, 1-2 days, 1-3 days, 1-4 days, 3-10 days, 3-7 days, 3-5 days, 5-10 days, and 5-7 days, inclusive. In some embodiments, the active agent is released from the drug reservoir as a continuous and/or sustained release over the application period.

### IV. EXAMPLES

The following examples are illustrative in nature and are in no way intended to be limiting

Unless otherwise specified, the following materials were used in the examples described below: the backing layer was SCOTCHPAK™ 9723; the release liner was a silicone-coated polyester (PET) film; the nonwoven tie layer was REEMAY® 2250; the rate controlling membrane was CELGARD® 2400 microporous polypropylene; and the contact adhesive layer was acrylate, polyisobutene (PIB), and/or silicone adhesive.

### Example 1

### Donepezil Transdermal Formulation

### Preparation of Drug Reservoir

An amount of 9 grams of EUDRAGIT® EPO (a proton accepting polymer) was dissolved in 74.71 grams of ethyl acetate. To the resulting solution, 6 grams of triacetin, 6 grams of glycerin, and 3 grams of SPAN® 20 (sorbitan monolaurate) were added and mixed. To the mixture, 12.6 grams of donepezil hydrochloride powder was dispersed. After addition of 7.8 grams of KOLLIDON® CL-M to the drug dispersed solution, the mixture was homogenized well. To the homogenized drug dispersion, 30.89 grams of DURO-TAK® 387-2287 (solid content 50.5%) was added and mixed well. The wet adhesive formulation was coated on a release liner and dried using a lab coater (Werner Mathis coater) to get a dry coat weight of 10 mg/cm².

### Preparation of Contact Adhesive

An amount of 4.00 grams of triacetin was mixed with 2.00 grams of sorbitan monolaurate and 34.52 grams of ethyl acetate. After addition of 8 grams of KOLLIDON® CL-M, the resulting mixture was homogenized. To the homogenized mixture, 51.48 grams of DURO-TAK® 387-2287 (solid content 50.5%) was added and mixed well. The wet adhesive formulation was coated on a release liner and dried to give a dry coat weight of 5 mg/cm².

### Lamination and Die-cut

CELGARD® 2400 rate controlling membrane or REEMAY® 2250 was laminated onto the adhesive side of the drug reservoir. Contact adhesive was then laminated on top of the CELGARD® 2400 membrane laminated with drug reservoir. The release liner on the drug reservoir side was replaced and laminated with backing film. The final five-layer laminate was die-cut into patches.

In vitro skin flux testing

Dermatomed human cadaver skin was obtained from a skin bank and frozen until ready for use. The skin was placed in water at 60° C. for 1-2 mins minute after thawing and the epidermis carefully separated from dermis. The epidermis was either used immediately or wrapped and frozen for later use.

In vitro skin flux studies were performed using a Franz type diffusion cell with an active diffusion area of 0.64 cm². The epidermis was mounted between the donor and receptor compartments of the diffusion cell. The transdermal delivery system was placed over the skin and the two compartments were clamped tight together.

The receptor compartment was filled with 0.01 M phosphate buffer, pH 6.5, containing 0.01% gentamicin. The solution in the receptor compartment was continually stirred using a magnetic stirring bar in the receptor compartment. The temperature was maintained at 32±0.5° C. Samples were drawn from the receptor solution at periodic intervals and the receptor solution was replaced with fresh phosphate buffers solution. Drug content in the samples was analyzed using HPLC for donepezil.

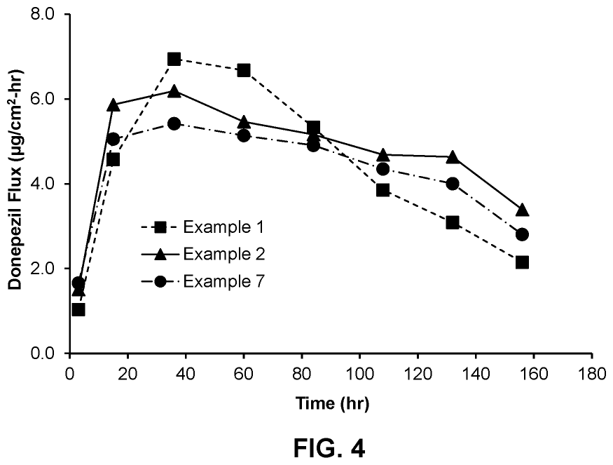

The results were calculated in terms of amount of drug diffused through the epidermis per cm² per unit time. Each data point is an average of four replica of diffusion cells. FIG. 4 shows the in vitro skin flux profile of donepezil formulated according to Example 1 (squares).

### Example 2

### Donepezil Transdermal Formulation

### Preparation of Drug Reservoir

An amount of 1.20 grams of SPAN® 20 was dissolved in a mixture of 6.00 g of triethyl citrate, and mixed with 1.80 grams of lauryl lactate and 89.69 grams of ethyl acetate. 6.00 grams of glycerin was added and mixed with the SPAN® 20 solution. To the mixture, 9.00 grams of donepezil hydrochloride and 1.82 grams of sodium bicarbonate were dispersed. After addition of 12.00 grams of KOLLIDON® CL-M to the drug dispersed solution, the mixture was homogenized well. To the homogenized drug dispersion, 43.93 grams of DURO-TAK® 387-2287 (solid content 50.5%) was added and mixed well. The wet adhesive formulation was coated on a release liner and dried using a lab coater (Werner Mathis coater) to get a dry coat weight of 12 mg/cm².

### Preparation of Contact Adhesive

An amount of 0.60 grams of SPAN® 20 (sorbitan monolaurate) was dissolved in 3.00 grams of triethyl citrate, and mixed with 0.9 grams of lauryl lactate, 25.45 grams of ethyl acetate, and 1.34 grams of isopropyl alcohol. After addition of 6.00 grams of KOLLIDON® CL-M, the mixture was homogenized. To the homogenized mixture, an amount of 38.61 grams of DURO-TAK® 387-2287 (solid content 50.5%) was added and mixed well. The wet adhesive formulation was coated on a release liner and dried to give a dry coat weight of 5 mg/cm².

### Lamination and Die-cut

CELGARD® 2400 rate controlling membrane or REEMAY® 2250 was laminated on adhesive side of the drug reservoir. Then contact adhesive was laminated on top of the CELGARD® 2400 membrane laminated with drug reservoir. The release liner on the drug reservoir side was replaced and laminated with backing film. The final five-layer laminate was die-cut into patches.

In vitro skin flux testing

The in vitro skin flux of donepezil from the transdermal system was measured as described in Example 1. FIG. 4 shows the in vitro skin flux profile of donepezil (triangles).

### Example 3

### Tamsulosin Transdermal Formulation

### Preparation of Drug Reservoir

An amount of 3.00 grams of EUDRAGIT® EPO was dissolved in 24.51 g of ethyl acetate. To the resulting solution, 2.00 grams of triethyl citrate, 2.00 grams of glycerin, and 1.00 grams of SPAN® 20 (sorbitan monolaurate) were added/mixed. To the mixture, 4.00 grams of tamsulosin hydrochloride powder was dispersed. After addition of 2.4 grams of KOLLIDON® CL-M to the drug dispersed solution, the mixture was homogenized well. To the homogenized drug dispersion, 11.09 grams of DURO-TAK® 387-2287 (solid content 50.5%) was added and well mixed. The wet adhesive formulation was coated on a release liner and dried using a lab coater (Werner Mathis coater) to get a dry coat weight of 10 mg/cm²

### Preparation of Contact Adhesive

An amount of 3.00 grams of triethyl citrate was mixed with 1.50 grams of sorbitan monolaurate (SPAN® 20) and 25.89 grams of ethyl acetate. After addition of 6.00 grams of KOLLIDON® CL-M, the mixture was homogenized. To the homogenized mixture, an amount of 38.61 grams of DURO-TAK 387-2287 (solid content 50.5%) was added and mixed well. The wet adhesive formulation was coated on a release liner and dried to give a dry coat weight of 5 mg/cm².

### Lamination and Die-cut

CELGARD® 2400 rate controlling membrane or REEMAY® 2250 was laminated on adhesive side of the drug reservoir. Then contact adhesive was laminated on top of CELGARD® 2400 membrane laminated with drug reservoir. The release liner on the drug reservoir side was replaced and laminated with backing film. The final five-layer laminate was die-cut into patches.

### Example 4

### Memantine Transdermal Formulation

### Preparation of Drug Reservoir

An amount of 18.00 grams of EUDRAGIT® EPO was dissolved in 14.53 g of ethyl acetate. To the resulting solution, 5.00 grams of triethyl citrate was added and mixed. To the resulting solution, 5.25 grams of memantine hydrochloride powder was added and well dispersed by homogenizing, and then 2.50 grams of glycerin were added and mixed. After addition of 1.75 grams of AEROSIL® 200P to the drug dispersed solution, the dispersion mixture was homogenized well. To the homogenized dispersion, 2.97 grams of DURO-TAK® 387-2287 (solid content 50.5%) was added and well mixed. The wet adhesive formulation was coated on a release liner and dried using a lab coater (Werner Mathis coater) to get a dry coat weight of 15 mg/cm².

### Preparation of Contact Adhesive

An amount of 3.00 grams of triethyl citrate was mixed with 17.26 grams of ethyl acetate. After addition of 4.00 grams of KOLLIDON® CL-M the mixture was homogenized. To the homogenized mixture, an amount of 25.74 grams of DURO-TAK® 387-2287 (solid content 50.5%) was added and mixed well. The wet adhesive formulation was coated on a release liner and dried to give a dry coat weight of 5 mg/cm².

### Lamination and Die-cut

CELGARD® 2400 rate controlling membrane was laminated to the drug reservoir. Then contact adhesive was laminated to the other side of CELGARD® 2400 membrane laminated with the drug reservoir. The release liner on the drug reservoir was replaced and laminated with backing film. The final five-layer laminate was die-cut into patches.

In vitro skin flux testing

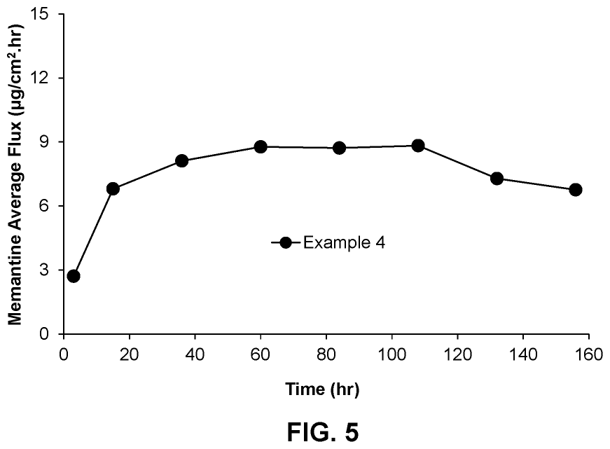

The in vitro skin flux of memantine from the transdermal system was measured as described in Example 1, where memantine content in the samples drawn from receptor solution were analyzed for memantine using LCMS. FIG. 5 shows the in vitro skin flux profile of memantine.

### Examples 5-8

### Additional Transdermal Formulations

Additional formulations were prepared according to the ingredients and quantities set forth in Table 1 and the general methods set forth in Example 1. FIG. 4 shows a flux profile of donepezil formulated according to the formulation identified as Example No. 7 in Table 1 below (circles).

TABLE 1

| | | Example No. → | | | | | | | |
|---|---|---|---|---|---|---|---|---|---|
| | | 1 | 2 | 3 | 4 | 5 | 6 | 7 | 8 |
| | | Composition (w/w %) | | | | | | | |
| Drug | Donepezil HCl | 21 | 15 | — | — | 15 | 25 | 24 | 25.2 |
| reservoir | Tamsulosin HCl | — | — | 20 | — | — | — | — | — |
| | Memantine HCl | | | | 21 | | | | |
| | Triethyl citrate | — | 10 | 10 | 20 | 10 | 10 | — | — |
| | Triacetin | 10 | — | — | — | — | — | 10 | — |
| | Sorbitan monolaurate | 5 | 2 | — | — | 5 | — | 5 | — |
| | Lauryl lactate | — | 3 | — | — | — | 6 | — | 5 |
| | Dimethyl succinate | — | — | — | — | — | — | — | 5 |
| | Glycerin | 10 | 10 | 10 | 10 | 10 | 10 | 10 | — |
| | Dimethylsulfoxide | — | — | — | — | — | — | — | 1 |
| | Eudragit EPO | 15 | — | 15 | 36 | — | 17.7 | 17 | 17.8 |
| | Sodium bicarbonate | — | 3.03 | — | — | 0.75 | — | — | — |
| | Kollidon CL-M | 13 | 15 | 12 | — | 20 | — | | — |
| | Aerosil 200P | | — | — | 7 | — | 7 | 7 | 7 |
| | Duro-Tak 387-2287 | 26 | 36.97 | 28 | 6 | 37.7 | | 27 | — |
| | Duro-Tak 387-2516 | — | — | — | — | — | 31.3 | — | 29 |
| RCM or tie | Celgard 2400 | yes | yes | yes | yes | — | yes | yes | yes |
| layer | Reemay 2250 | — | — | — | — | yes | — | — | — |
| Contact | Triethyl citrate | — | 10 | 10 | 15 | 10 | 10 | — | — |
| adhesive | Triacetin | 10 | — | — | — | — | — | 10 | — |
| | Sorbitan monolaurate | 5 | 2 | 5 | — | 5 | — | 5 | — |
| | Lauryl lactate | — | 3 | — | — | — | 6 | — | 5 |
| | Dimethyl succinate | — | — | — | — | — | — | — | 5 |
| | Dimethyl sulfoxide | — | — | — | — | — | — | — | 1 |
| | Kollidon CL-M | 20 | 20 | 20 | 20 | 20 | 20 | — | — |
| | Aerosil 200P | | — | — | — | — | — | 7 | 7 |
| | Duro-Tak 387-2287 | 65 | 65 | 65 | 65 | 65 | 64 | 78 | — |
| | Duro-Tak 387-2516 | — | — | — | — | — | — | — | 82 |

| | | | | | | | | |
|---|---|---|---|---|---|---|---|---|
| 168 hour average flux | 4.88 | 5.09 | N/A | 8.16 | 8.16 | 4.61 | 4.58 | 3.25 |
| (μg/cm²-hr) | | | | | | | | |

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

All patents, patent applications, patent publications, and other publications mentioned herein are hereby incorporated by reference in their entirety. Where a patent, application, or publication contains express definitions, those definitions should be understood to apply to the incorporated patent, application or publication in which they are found and not to the present application unless otherwise indicated.

It is claimed:

1. A composition for transdermal delivery, comprising:
a drug reservoir comprising a salt form of an active agent and a proton accepting and/or proton donating entity.

2. The composition of claim 1, wherein the drug reservoir comprises an amine salt form of an active agent and a proton accepting entity.

3. The composition of claim 1, wherein the active agent is donepezil, rivastigmine, memantine, or tamsulosin.

4. The composition of claim 1, wherein the drug reservoir comprises about 1% to about 70% w/w of the active agent.

5. The composition of claim 2, wherein the proton accepting entity is a proton accepting polymer. 6 The composition of claim 2, wherein the proton accepting entity is an amine functionalized polystyrene miscrosphere or a dimethyl aminoethyl methacrylate-based acrylate.

7. The composition of claim 2, wherein the drug reservoir comprises about 0.5% to about 35% w/w of the proton accepting entity.

8. The composition of claim 1, wherein the drug reservoir comprises an acid salt form of an active agent and a proton donating polymer.

9. The composition of claim 8, wherein the active agent is an acid salt drug selected from the group consisting of sodium alendronate, tresprostinil sodium, sodium diclofenac, naproxen sodium, and ketoprofen sodium.

10. The composition of claim 8, wherein the drug reservoir comprises about 5% to about 35% w/w of the active agent.

11. The composition of claim 8, wherein the proton donating polymer is an anionic copolymer based on methacrylic acid or a carboxylated polystyrene microsphere.

12. The composition of claim 8, wherein the drug reservoir comprises about 0.5% to about 35% w/w of the proton donating polymer.

13. The composition of claim 1, further comprising a salt form solubilizer selected from the group consisting of water, alcohols, glycerol, propylene glycol, ethylene glycol, dimethyl sulfoxide, and N-methylpyrrolidone.

14. The composition of claim 13, wherein the drug reservoir comprises up to 15% w/w of the salt form solubilizer.

15. The composition of claim 1, further comprising a neutral form solubilizer selected from the group consisting of a fatty acid ester, a dicarboxylic acid ester, a glycerol ester, a lactate, a fatty alcohol, sorbitan monolaurate, sorbitan monooleate, lauryl lactate, propylene glycol monolaurate, dimethyl succinate, lauryl alcohol, and oleyl alcohol.

16. The composition of claim 15, wherein the drug reservoir comprises up to 15% w/w of the neutral form solubilizer.

17. The composition of claim 1, further comprising a plasticizer selected from the group consisting of a dicarboxylic acid ester, an adipate, a sebacate, a maleate, a tricarboxylic ester, triethyl citrate, tributyl citrate, a glycerol esters, and triacetin.

18. The composition of claim 17, wherein the drug reservoir comprises up to 20% w/w of the plasticizer.

19. The composition of claim 1, further comprising an additive selected from the group consisting of crospovidone and colloidal silicone dioxide.

20. The composition of claim 19, wherein the drug reservoir comprises up to 25% w/w of the additive.

21. The composition of claim 1, wherein the drug reservoir comprises an adhesive agent selected from the group consisting of an acrylate, polyisobutylene, silicone adhesive, and styrene block copolymer based adhesive.

22. The composition of claim 21, wherein the drug reservoir comprises up to 65% w/w of the adhesive agent.

23. A transdermal patch comprising a drug reservoir layer comprising a composition of claim 1; a backing layer; and a contact adhesive layer.

24. The transdermal patch of claim 23, wherein the backing layer is an occlusive polymer film.

25. The transdermal patch of claim 23, wherein the contact adhesive layer comprises an adhesive selected from the group consisting of an acrylate, polyisobutylene, silicone adhesive, and styrene block copolymer based adhesive.

26. The transdermal patch of claim 23, further comprising a nonwoven tie layer between the drug reservoir and the contact adhesive layer.

27. The transdermal patch of claim 23, further comprising a rate-controlling membrane between the drug reservoir layer and the contact adhesive layer.

28. The transdermal patch of claim 23, wherein the patch comprises at least two drug reservoir layers.

29. The transdermal patch of claim 28, wherein each of the at least two drug reservoir layers is separated by a nonwoven tie layer.

30. The transdermal patch of claim 28, wherein each of the at least two drug reservoir layers is separated by a rate-controlling membrane.

31. A method of transdermally administering an active agent to a patient in need thereof, comprising providing a composition according to claim 1 to a patient in need thereof.

32. A method for treating Alzheimer's disease, Parkinson's disease, attention deficit hyperactivity disorder, narcolepsy, depression, anxiety disorder, obsessive compulsive disorder, opioid dependence, benign prostatic hyperplasia, or acute urinary retention comprising providing a composition according to claim 1 to a patient in need thereof.

33. The method of claim 31, further comprising administering or instructing to administer to the skin of the patient the composition.

34. The method of claim 33, wherein said administering achieves a therapeutically effective blood concentration of the active agent.

35. A method of transdermally administering an active agent to a patient in need thereof, comprising providing a transdermal patch according to claim 23 to a patient in need thereof.

36. A method for treating Alzheimer's disease, Parkinson's disease, attention deficit hyperactivity disorder, narcolepsy, depression, anxiety disorder, obsessive compulsive disorder, opioid dependence, benign prostatic hyperplasia, or acute urinary retention comprising providing a transdermal patch according to claim 23 to a patient in need thereof.

37. The method of claim 35, further comprising administering or instructing to administer to the skin of the patient the transdermal patch.

38. The method of claim 37, wherein said administering achieves a therapeutically effective blood concentration of the active agent.
